(19) United States
(12) Patent Application Publication
(10) Pub. No.: US 2010/0152100 A1
(43) Pub. Date: Jun. 17, 2010

(54) ACCELERATED THERAPY

(75) Inventors: William McCulloch, Raleigh, NC (US); Henry Miles Prince, Elsternwick (AU)

Correspondence Address: CHOATE, HALL & STEWART LLP, TWO INTERNATIONAL PLACE, BOSTON, MA, 02110, US

(73) Assignees: Gloucester Pharmaceuticals, Inc.; Peter Maccallum Cancer Centre

(21) Appl. No.: 12/512,419

(22) Filed: Jul. 30, 2009

Related U.S. Application Data
(60) Provisional application No. 61/084,797, filed on Jul. 30, 2008.

(51) Int. Cl. A61K 38/15; A61P 1/00; A61P 9/00

(52) U.S. Cl. 514/10

(57) ABSTRACT

The present invention encompasses the surprising finding that romidepsin can safely be administered to humans on an accelerated dosing schedule.

### CROSS REFERENCE TO RELATED APPLICATIONS

The present application is copending with, shares at least one common inventor with and claims priority to U.S. provisional patent application Ser. No. 61/084,797, filed Jul. 30, 2008, the entire contents of which are incorporated herein by reference.

### BACKGROUND OF THE INVENTION

Many chemotherapeutic agents display severe, typically dose-limiting toxicities. Indeed, many agents that demonstrate potent cytotoxicity in vitro and therefore appear to be promising anti-cancer agents cannot ultimately be used in therapy because of their severe toxicities.

Both the scientific and popular literature are replete with examples of chemotherapeutic agents that, when administered incorrectly (e.g., at too high a dose) produce serious, if not devastating side effects.

### SUMMARY OF THE INVENTION

The present invention encompasses the finding that romidepsin can be administered to subjects intravenously on an accelerated dosing schedule. In particular, the present invention encompasses the finding that romidepsin can be administered to subjects intravenously so that individual unit doses within a dosing schedule are administered over a time period that is less than about one hour. Remarkably, despite the potent cytotoxicity of romidepsin, the present inventors have found that such accelerated dosing can be performed without increasing the rate of serious adverse events as compared with the standard rate observed when individual unit doses are administered over a time period of about 4 hours. Thus, according to the present invention, a four-fold increase in rate of intravenous administration of a unit dose of romidepsin can be achieved without material increase in risk to patients.

Among other things, therefore, the present invention provides accelerated dosing methods that reduce the time and hassle associated with administration of romidepsin.

### DEFINITIONS

Adverse event: The term “adverse event” as used herein has its art understood meaning and refers to any untoward medical occurrence in a patient or clinical investigation subject administered a pharmaceutical product. An adverse event does not necessarily have to have a causal relationship with the treatment administered.

Adverse reaction: The term “adverse reaction” as used herein had its art understood meaning and refers to any noxious and unintended responses to a medicinal product related to any dose.

Cell Proliferative Disorder, Disease, or Condition: The term “cell proliferative disease or condition” is meant to refer to any condition characterized by aberrant cell growth, preferably abnormally increased cellular proliferation.

Combination Therapy: The term “combination therapy”, as used herein, refers to those situations in which two or more different pharmaceutical agents are administered in overlapping regimens so that the subject is simultaneously exposed to both agents.

Electrolyte: In general, the term “electrolyte”, as used herein, refers to physiologically relevant free ions. Representative such free ions include, but are not limited to sodium (Na⁺), potassium (K⁺), calcium (Ca²⁺), magnesium (Mg²⁺) chloride (Cl⁻), phosphate (PO4³⁻), and bicarbonate (HCO₃⁻).

DAC Inhibitor: In general, any agent that specifically inhibits a deacetylase is considered to be a DAC inhibitor. Any agent that specifically inhibits a histone deacetylase is considered to be an HDAC inhibitor. Any agent that specifically inhibits a tubulin deacetylase is considered to be a TDAC inhibitor. Those of ordinary skill in the art will appreciate that, unless otherwise set forth herein or known in the art, DAC inhibitors may be administered in any form such as, for example, salts, esters, prodrugs, etc. Furthermore, DAC inhibitors that contain chiral centers may be administered as single stereoisomers or as mixtures, including racemic mixtures, so long as the single stereoisomer or mixture has DAC inhibitor activity.

DAC Inhibitor Therapy: As used herein, the phrase “DAC inhibitor therapy” refers to a regimen by which a DAC inhibitor is administered to an individual. Commonly, DAC inhibitor therapy will involve administration of multiple individual unit doses of a DAC inhibitor, spaced out over time. Such individual doses may be of different amounts or of the same amount. Furthermore, those of ordinary skill in the art will readily appreciate that different dosing regimens (e.g., number of doses, amount(s) of doses, spacing of doses) are typically employed with different DAC inhibitors.

Dosing Regimen: A “dosing regimen”, as that term is used herein, refers to a set of unit doses (typically more than one) that are administered individually separated by periods of time. The recommended set of doses (i.e., amounts, timing, route of administration, etc.) for a particular pharmaceutical agent constitutes its dosing regimen.

Initiation: As used herein, the term “initiation” when applied to a dosing regimen can be used to refer to a first administration of a pharmaceutical agent to a subject who has not previously received the pharmaceutical agent. Alternatively or additionally, the term “initiation” can be used to refer to administration of a particular unit dose of a pharmaceutical agent during therapy of a patient.

Pharmaceutical agent: As used herein, the phrase “pharmaceutical agent” refers to any agent that, when administered to a subject, has a therapeutic effect and/or elicits a desired biological and/or pharmacological effect.

Pharmaceutically acceptable carrier or excipient: As used herein, the term “pharmaceutically acceptable carrier or excipient” means a non-toxic, inert solid, semi-solid or liquid filler, diluent, encapsulating material or formulation auxiliary of any type.

Pharmaceutically acceptable ester: As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

Pharmaceutically acceptable prodrug: The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

Pharmaceutically acceptable salt: As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Romidepsin: The term “romidepsin” refers to a compound of the chemical structure:

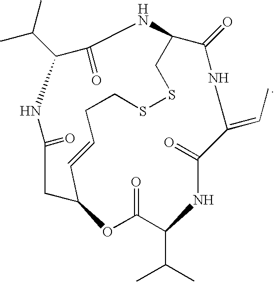

Romidepsin is also known in the art by the names FK228, FR901228, NSC630176, or depsipeptide. The identification and preparation of romidepsin is described in U.S. Pat. No. 4,977,138, which is incorporated herein by reference. Additional methods of preparing romidepsin are described in WO08/083,288 and WO08/083,290, which are incorporated herein by reference. The molecular formula is C₂₄H₃₆N₄O₆S₂; and the molecular weight is 540.71. Certain crystalline forms of romidepsin are disclosed in U.S. Pat. Pub. 2008/0227975, which is incorporated herein by reference. Romidepsin has the chemical name, (1S,4S,10S,16E,21R)-7-[(2Z)-ethylidene]-4,21-diisopropyl-2-oxa-12,13-dithia-5,8,20,23-tetraazabicyclo[8.7.6]tricos-16-ene-3,6,9,19,22-pentanone. Romidepsin has been assigned the CAS number 128517-07-7. In crystalline form, romidepsin is typically a white to pale yellowish white crystal or crystalline powder. Any of a variety of forms of romidepsin may be utilized in accordance with the present invention. For example, romidepsin may be provided in a salt form (e.g., as a pharmaceutically acceptable salt of the compound). Alternatively or additionally, romidepsin may be provided and/or administered as in a pro-drug, ester, or protected form, etc.

Serious adverse event: The term “serious adverse event”, as used herein, has its art-understood meaning and refers to any untoward medical occurrence that at any dose, for example, results in death, is life threatening, requires inpatient hospitalization (or prolongation of existing hospitalization), results in persistent or significant disability or incapacity (defined as a substantial disruption of a patient's ability to carry out normal life functions), etc. In some embodiments, a serious adverse event is a “serious adverse drug experience”, as that term is used by the United States Food and Drug Administration, for example as defined in 21 CFR §310.305(b), which says that a serious adverse event is any adverse drug experience occurring at any dose that results in any of the following outcomes: death, a life-threatening adverse drug experience, inpatient hospitalization or prolongation of existing hospitalization, a persistent or significant disability/incapacity, or a congenital anomaly/birth defect. Important medical events that may not result in death, be life-threatening, or require hospitalization may be considered a serious adverse drug experience when, based upon appropriate medical judgment, they may jeopardize the patient or subject and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

Susceptible to: The term “susceptible to” is used herein to refer to an individual having higher risk (typically based on genetic predisposition, environmental factors, personal history, or combinations thereof) of developing a particular disease or disorder, or symptoms thereof, than is observed in the general population.

Therapeutically effective amount: The term “therapeutically effective amount” of an pharmaceutical agent or combination of agents is intended to refer to an amount of agent(s) which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). A therapeutically effective amount is commonly administered in a dosing regimen that may comprise multiple unit doses. For any particular pharmaceutical agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific pharmaceutical agent employed; the duration of the treatment; and like factors as is well known in the medical arts.

Treatment: As used herein, the term “treatment” (also “treat” or “treating”) refers to any administration of a pharmaceutical agent that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

Unit dose: The term “unit dose”, as used herein, refers to a discrete administration of a pharmaceutical agent, typically in the context of a dosing regimen.

### DETAILED DESCRIPTION OF CERTAIN EMBODIMENTS OF THE INVENTION

### Standard Romidepsin Dosing

Romidepsin has been studied in a variety of clinical trials. Intravenous dosing regimens that have been studied include daily dosing for 2 weeks, twice weekly dosing for 4 weeks, thrice weekly dosing for 4 weeks, and various other intermittent schedules (e.g., on days 1, 3, and 5; on days 4 and 10; on days 1, 8 and 15; on days 1 and 15; on days 5 and 12; or on days 5, 12, and 19 of 21 or 28 day cycles).

Typical unit doses of romidepsin are within the range of about 0.5 mg/m² to about 28 mg/m² body surface area, and often within the range of about 6 to about 18 mg/m². Romidepsin is commonly administered in unit doses with in the range of about 10 mg/m² to about 17 mg/m². Particular common unit doses are 13 mg/m², 14 mg/m², and 15 mg/m².

As of March 2008, the standard intravenous regimen for romidepsin that is utilized in most trials involves administrations of individual unit doses over 4 hours on days 1, 8, and 15, with courses repeating every 28 days. Often, several courses (e.g., at least 4, at least 6, or more) are administered. Indeed, instances have been reported of as many as 72 courses being administered.

Early clinical and preclinical studies of romidepsin found that toxicities associated with romidepsin were increased for more rapid administration regimens. For example, both general toxicity and cardiotoxicity were found to be more significant for rapid injection (e.g., 30-second bolus or 10-minute injection) than for 4-hour infusion (see, for example, (see Shah et al., Cardiotoxicity of Histone Deacetylase Inhibitor Depsipeptide in Patients with Metastatic Neuroendocrine Tumors, Clin. Cancer Res. 12:3997, 2006, which states at page 4002, left column, first full paragraph, line 17:
“Subsequent preclinical studies conducted by NCI in dogs and mice showed that intermittent (q4 days×3) schedule was better tolerated than daily dosing. Furthermore, toxicity in general and cardiotoxicity specifically, seemed to be related to the rate of administration; 4-hour infusion was much better tolerated than a 30-second bolus or 10-minute injection. Based on this preclinical experience, an intermittent dosing schedule delivered as a 4-hour i.v. infusion was found to be optimum and was used for initial phase I clinical trials”).
   In most standard intravenous romidepsin dosing regimens, individual unit doses are administered by 4 hour infusion.

### Accelerated Dosing

The present invention provides accelerated dosing regimens for romidepsin in which one or more individual unit doses is administered intravenously over a period of time that is less than or equal to about one hour. In some embodiments, one or more individual doses is administered intravenously over a period of time that is less than about 50 minutes, 40 minutes, 30 minutes, 20 minutes, or less. Any regimen that includes at least one unit dose administered over a period of time that is less than about one hour (60 minutes) may be considered an accelerated dosing regimen in accordance with the present invention.

In some embodiments of the present invention, all unit doses within a regimen are administered intravenously over a time period that is less than or equal to about one hour. In some embodiments, only some of the unit doses within a regimen are administered over a time period that is less than or equal to about one hour. In some embodiments, one or more unit doses within a regimen are administered by a route other than intravenous administration (e.g., oral, subcutaneous, nasal, topical, etc).

The present invention demonstrates that accelerated dosing regimens of romidepsin can be administered without a significant increase in toxicity or adverse events, and particularly in serious adverse events, as compared with a comparable regimen (e.g., an otherwise identical regimen) in which individual unit doses are administered intravenously over a 4-hour period.

The present invention particularly demonstrates that accelerated dosing regimens can be administered without a significant increase in toxicity or adverse events, and particularly in serious adverse events, as compared with a standard regimen of romidepsin administered by 4-hour intravenous infusion of a dose of about 6-14 mg/m² on days 1, 8, and 15 of a 28 day cycle.

In some embodiments of the present invention, romidepsin is administered in an accelerated dosing regimen that is identical to a standard dosing regimen (see above) except that one or more unit doses is administered over a time period that is less than about 1 hour (e.g., rather than over a time period of about 4 hours).

Typical unit doses of romidepsin for use in accordance with the present invention are within the range of about 0.5 mg/m² to about 28 mg/m². In certain embodiments, unit doses are in the range of about 1 mg/m² to about 25 mg/m². In certain embodiments, unit doses are in the range of about 0.5 mg/m² to about 15 mg/m². In certain embodiments, unit doses are the range of about 1 mg/m² to about 15 mg/m². In certain embodiments, unit doses are in the range of about 1 mg/m² to about 8 mg/m². In certain embodiments, unit doses are in the range of about 0.5 mg/m² to about 5 mg/m². In certain embodiments, the unit doses are in the range of about 2 mg/m² to about 10 mg/m². In some embodiments, unit doses are in the range of about 10 mg/m² to about 20 mg/m². In certain embodiments, unit doses are in the range of about 5 mg/m² to about 10 mg/m². In some embodiments, unit doses are in the range of about 10 mg/m² to about 15 mg/m². In some embodiments, unit doses are in the range of about 6 to about 19 mg/m². In some embodiments, unit doses are approximately 8 mg/m². In still other embodiments, the unit doses are approximately 9 mg/m². In still other embodiments, unit doses are approximately 10 mg/m². In still other embodiments, unit doses are approximately 11 mg/m². In still other embodiments, unit doses are approximately 12 mg/m². In still other embodiments, unit doses are approximately 13 mg/m². In still other embodiments, unit doses are approximately 14 mg/m². In still other embodiments, unit doses are approximately 15 mg/m². In still other embodiments, unit doses are approximately 30 mg/m².

In certain embodiments, different individual unit doses within a romidepsin therapy regimen are different. For example, in some embodiments, increasing doses of romidepsin are administered over the course of a cycle. To give but one such example, in certain embodiments, a dose of approximately 8 mg/m² is administered, followed by a dose of approximately 10 mg/m², followed by a dose of approximately 12 mg/m² may be administered over a cycle.

As will be appreciated by one of skill in the art, the amount of romidepsin administered in individual unit doses may vary depending on the form of romidepsin being administered. The dosages given herein are dose equivalents with respect to the active ingredient, romidepsin.

In certain embodiments of the invention, individual unit doses of romidepsin are administered on one day followed by several days on which romidepsin is not administered. In certain embodiments, romidepsin is administered twice a week. In certain embodiments, romidepsin is administered once a week. In other embodiments, romidepsin is administered every other week.

To give but a few examples of appropriate dosing schedules for use in accordance with the present invention, romidepsin may be administered daily (for example for 2 weeks), twice weekly (for example for 4 weeks), thrice weekly (for example for 4 weeks), or on any of a variety of other intermittent schedules (e.g., on days 1, 3, and 5; on days 4 and 10; on days 1 and 15; on days 5 and 12; or on days 5, 12, and 19 of 21 or 28 day cycles).

In certain embodiments, romidepsin is administered on days 1, 8, and 15 of a 28 day cycle. In certain particular embodiments, an 8 mg/m² dose of romidepsin is administered on day 1, a 10 mg/m² dose of romidepsin is administered on day 8, and a 12 mg/m² dose of romidepsin is administered on day 15. In certain embodiments, romidepsin is administered on days 1 and 15 of a 28 day cycle with day 8 being skipped. A 28 day dosing cycle may be repeated. In certain embodiments, a 28 day cycle is repeated 2-10, 2-7, 2-5, or 3-10 times. In certain embodiments, the treatment includes 5 cycles. In certain embodiments, the treatment includes 6 cycles. In certain embodiments, the treatment includes 7 cycles. In certain embodiments, the treatment includes 8 cycles. In certain embodiments, 10 cycles are administered. In certain embodiments, greater than 10 cycles are administered.

As noted above, in certain embodiments of the present invention, one or more unit doses within a romidepsin dosing regimen may be administered via a route other than intravenous administration. To give but one example, one or more doses may be administered orally. In certain embodiments, romidepsin is dosed orally in the range of 10 mg/m² to 300 mg/m². In certain embodiments, romidepsin is dosed orally in the range of 25 mg/m² to 100 mg/m². In certain embodiments, romidepsin is dosed orally in the range of 100 mg/m² to 200 mg/m². In certain embodiments, romidepsin is dosed orally in the range of 200 mg/m² to 300 mg/m². In certain embodiments, romidepsin is dosed orally at greater than 300 mg/m². In certain embodiments, romidepsin is dosed orally in the range of 50 mg/m² to 150 mg/m². In other embodiments, the oral dosage ranges from 25 mg/m² to 75 mg/m².

In certain embodiments, romidepsin is administered orally on a daily basis. In some embodiments, romidepsin is administered orally every other day. In still other embodiments, romidepsin is administered orally every third, fourth, fifth, or sixth day. In certain embodiments, romidepsin is administered orally every week. In certain embodiments, romidepsin is administered orally every other week.

In some embodiments, one or more unit doses of romidepsin is administered topically.

As will be appreciated by one of skill in the art, the dosage, timing and/or routes of administration of particular unit doses of romidepsin may vary depending on the patient and condition being treated. In certain embodiments, the cycles are continued as long as the patient is responding. Therapy may be terminated once there is disease progression, a cure or remission is achieved, or side effects become intolerable. Adverse side effects may also call for lowering the dosage of romidepsin administered, or for adjusting the schedule by which doses are administered.

Toxicity and Adverse Events with Romidepsin

As noted above, romidepsin has been administered to patients in a variety of different clinical contexts and studies. In general, observed toxicities include fatigue, nausea, vomiting, and myelosuppression (usually thrombocytopenia and/or neutropenia, e.g., Grade 3). Non-specific S-T segment changes on ECG occur in many patients, and prolongation of QTc intervals can also be seen. Typically, observed toxicities were mild to moderate. Observed changes in ECGs typically did not correlate with elevated serial serum troponin levels and multiple gated acquisition (MUGA) scans, both of which were consistently normal.

In early development, 6 deaths occurred (out of more than 450 patients) during clinical investigations of romidepsin. In all but one of the deaths, significant cardiovascular risk factors were either present at the time of entry into the romidepsin study or appeared during the course of the study. The sixth patient had a history of sarcoidosis and was simultaneously administered another drug that also is known to cause QTc prolongation.

Hematologic Events

Neutropenia and/or thrombocytopenia are sometimes observed in patients receiving romidepsin. It is generally recommended that further treatment be withheld from patients with Grade 3 or Grade 4 neutropenia or thrombocytopenia, until their specific cytopenia returns to Grade 1 (i.e., ANC recovered to >1.9×10⁹/L and platelet count recovered to ≧75×10⁹/L) or below, at which point therapy can be continued at full dose. If Grade 4 neutropenia or thrombocytopenia lasting more than 5 days or associated with bleeding is observed, then it is generally recommended that treatment be withheld until specific cytopenia returns to Grade 1 or below, at which point therapy can continue, preferably at a reduced dose (e.g., 10 mg/m²). If Grade 4 febrile (≧38.5° C.) neutropenia or thrombocytopenia that requires platelet transfusion is observed, it is generally recommended that treatment be withheld until the specific cytopenia returns to Grade 1 or below, at which point therapy can continue, preferably at a reduced dose (e.g., 10 mg/m²).

Hematologic events are typically observed at a rate of about 21-52% with standard romidepsin dosing regimens (National Cancer Institute IND 57,810 Annual Report, 2007). For example, the NCI-2007 Annual Report provides the following rates for the following blood and bone marrow abnormalities: platelets (52%), hemoglobin/anemia (41%), abnormal white blood cell count (39%), abnormal ANC/AGC (37%), and lymphopenia (21%)(National Cancer Institute INB 57,810 Annual Report, 2007).

Cardiac Events

Cardiac events observed with romidepsin dosing can include any or all of, for example:
Prolongation of QTc to ≧500 msec or an increase of ≧60 msec from pretreatment baseline for the current treatment cycle
Ventricular arrhythmia (i.e., ventricular tachycardia or ventricular fibrillation [≧3 beats in a row])
Sinus tachycardia (pulse >140/min after recumbency)
New occurrence of atrial dysrhythmias (SVT, atrial fibrillation, or atrial flutter), ST and T-wave changes indicative of repolarization abnormalities or ischemia (e.g., ST depression of ≧2 mm [measured from isoelectric line to ST segment] and/or T-wave inversion of ≧4 mm [measured from isoelectric line to peak of T-wave] as long as the main QRS vector is positive).
   The literature reports that the median change in QTc from baseline is 16.5 milliseconds (see Piekarz et al., Clin Cancer Res 12:3762, 2006). Table 1 presents common recommendations for dose modification when cardiac events are observed:

TABLE 1

| Parameters/Symptoms | Change | Action | Dosing/Continuation |
|---|---|---|---|
| Sinus tachycardia | Pulse >140/min after | Hold further dosing, | If resolved, restart |
| | recumbancy | consult local cardiologist, | treatment, preferably at a |
| Atrial dysrhythmia (SVT, | New occurrence | and treat appropriately | reduced dose (e.g., 10 |
| atrial fibrillation, or atrial | | | mg/m²) |
| flutter) | | | If not resolved, |
| Prolongation of QTcf | To ≧500 msec | | discontinue therapy |
| compared to pre-treatment | OR | | |
| baseline in a treatment | Increase by ≧60 msec | | |
| cycle | | | |
| Ventricular tachycardia | ≧3 beats in a row | | |
| Ventricular fibrillation | New occurrence | Hold further dosing and | Hold further dosing until |
| | | treat appropriately. The | medical monitor and |
| | | medical monitor should be | cardiologist evaluation is |
| | | notified and local | complete |
| | | cardiologist should be | |
| | | consulted | |
| A subsequent episode of any of the above, despite dose reduction | | | Discontinue romidepsin |
| | | | administration |
| T-wave morphology | Inversion of ≧4 mm^{a} | Hold further dosing, | If resolved, restart |
| ST-segment | Depression of ≧2 mm^{b} | consult local cardiologist, | treatment, preferably at a |
| | | and treat appropriately | reduced dose (e.g., 10 |
| | | | mg/m²) |
| | | | In some patients, ST |
| | | | segment and T-wave |
| | | | morphology changes may |
| | | | recur despite a dose |
| | | | reduction. In such cases, |
| | | | further treatment should be |
| | | | withheld until the ECG |
| | | | changes resolve. If the |
| | | | patient experiences no |
| | | | concomitant clinical |
| | | | events, treatment may be |
| | | | resumed, preferably at the |
| | | | reduced dose level. |
| | | | If not resolved, |
| | | | discontinue therapy. |

^{a}Measured from isoelectric line to peak of T-wave
^{b}Measured from isoelectric line to ST segment

Cardiac events are typically observed at a rate of about 24% with standard romidepsin dosing regimens (National Cancer Institute IND 57,810 Annual Report, 2007)

Gastrointestinal Events

Gastrointestinal events are typically observed at a rate of about 15-64% with standard romidepsin dosing regimens (National Cancer Institute IND 57,810 Annual Report, 2007). For example, the NCI-2007 Annual Report provides the following rates for the following gastrointestinal events: nausea (64%), anorexia (39%), vomiting (39%), constipation (19%), dysguesia (18%), and diarrhea (15%) (National Cancer Institute INB 57,810 Annual Report, 2007).

The present invention demonstrates that romidepsin can be administered via accelerated dosing regimens without a clinically significant increase in relevant toxicities (e.g., in the rate and/or severity of one or more of dose limiting toxicities, serious adverse events, and/or adverse events). For example, in some embodiments, the present invention provides accelerated dosing regimens for romidepsin in which the rate of observed toxicities (e.g., fatigue, hematological toxicities, cardiac toxicities, gastrointestinal toxicities, constitutional toxicities, or a combination thereof) is not materially worse than that observed for administration of a comparable dosing regimen that differs only in that unit doses of romidepsin are administered intravenously over a time period of about 4 hours. In some embodiments, the present invention provides accelerated dosing regimens for romidepsin in which the rate of observed toxicities is not materially worse than that observed for administration of a standard romidepsin therapy regimen.

In some embodiments, the present invention provides accelerated dosing regimens for romidepsin in which the subject receiving romidepsin does not suffer one or more particular adverse events, or serious adverse events, within a designated time period. In some embodiments, the designated time period is during administration of the accelerated dose. In some embodiments, the designated time period is within about 2 to about 6 hours after the end of infusion of the accelerated dose. In some embodiments, the designated time period is within about 2, 4, 6, 8, 10, 12, 14, 16, 18, 20, 22, 24, 26, 28, 30, 32, 34, 36, 38, 40 42, 44, 46, 48 or more hours after the end of infusion of the accelerated dose.

Any side effect, toxicity, or adverse event (including adverse events) may be absent from the designated time period. To give but a few examples, in some embodiments, the subject subject's QTc remains below about 500 msec during the designated time period; in some embodiments, the subject does not suffer a ventricular arrhythmia during the designated time period; in some embodiments, the subject does not suffer sinus tachycardia during the designated time period; in some embodiments, the subject does not suffer an atrial dysrhythmia during the designated time period; in some embodiments the subject does not suffer ST or T-wave changes indicative of repolarization during the designated time period.

### Combination Therapy

According to the present invention, romidepsin may be administered in combination with one or more other pharmaceutical agents. For example, romidepsin may be administered in combination with one or more other chemotherapeutic agents and/or in combination with one or more other pharmaceutical agents (e.g., pain relievers, anti-inflammatories, antibiotics, steroidal agents, anti-folates, kinase inhibitors, methyl transferase inhibitors, antibodies, etc).

In certain embodiments, romidepsin is administered in combination with one or more cytotoxic agents. Exemplary such cytotoxic agents include, for example, gemcitabine, decitabine, and flavopiridol. Alternatively or additionally, romidepsin may be administered in combination with one or more taxanes and/or one or more proteasome inhibitors. Exemplary such proteasome inhibitors include, for example, bortezomib (VELCADE®), peptide boronates, salinosporamide A (NPI-0052), lactacystin, epoxomicin (Ac(Me)-Ile-Ile-Thr-Leu-EX), MG-132 (Z-Leu-Leu-Leu-al), PR-171, PS-519, eponemycin, aclacinomycin A, CEP-1612, CVT-63417, PS-341 (pyrazylcarbonyl-Phe-Leu-boronate), PSI (Z-Ile-Glu(OtBu)-Ala-Leu-al), MG-262 (Z-Leu-Leu-Leu-bor), PS-273 (MNLB), omuralide (clasto-lactacystin-β-lactone), NLVS (Nip-Leu-Leu-Leu-vinyl sulfone), YLVS (Tyr-Leu-Leu-Leu-vs), dihydroeponemycin, DFLB (dansyl-Phe-Leu-boronate), ALLN (Ac-Leu-Leu-Nle-al), 3,4-dichloroisocoumarin, 4-(2-aminoethyl)-benzenesulfonyl fluoride, TMC-95A, gliotoxin, EGCG ((−)-epigallocatechin-3-gallate), YU101 (Ac-hFLFL-ex), and combinations thereof.

In certain embodiments, romidepsin is administered in combination with one or more anti-folates. For example, in some such embodiments, romidepsin is administered in combination with one or more of: folinic acid (leucovorin), methotrexate, pralatrexate, premextred, triazinate, and combinations thereof.

In certain embodiments, romidepsin is administered in combination with one or more kinase inhibitors (e.g., tyrosine kinase inhibitors). For example, in some such embodiments, romidepsin is administered in combination with one or more antibodies that act as a kinase inhibitor. In some embodiments, romidepsin is administered in combination with one or more of ABT-869, AC220, AZD7762, BIBW 2992, BMS-690154, CDKIAT7519, CYC116, ISIS3521, GSK690693, GSK-461364, MK-0457, MLN8054, MLN8237, MP470, ON 01910.Na, OSI-930, PHA-739358, R935788, SNS-314, TLN-232, XL147, XL228, XL281, XL418, and/or XL765.

In certain embodiments, romidepsin is administered in combination with one or more methyl transferase inhibitors.

In certain embodiments, romidepsin is administered in combination with one or more therapeutic antibodies. For example, in some such embodiments, romidepsin is administered in combination with one or more of: bevacizumab, cetuximab, dasatinib, erlotinib, geftinib, imatinib, lapatinib, nilotinib, panitumumab, pegaptanib, ranibizumab, sorafenib, sunitinib, trastuzumab, or any antibody that binds to an antigen bound by one of these.

In some embodiments, romidepsin is administered in combination with an anti-inflammatory agent such as aspirin, ibuprofen, acetaminophen, etc., pain reliever, anti-nausea medication, or anti-pyretic.

In certain embodiments, romidepsin is administered in combination with a steroidal agent. For example, in certain embodiments, romidepsin is administered in combination with a steroidal agent selected from the group consisting of alclometasone diproprionate, amcinonide, beclomethasone diproprionate, betamethasone, betamethasone benzoate, betamethasone diproprionate, betamethasone sodium phosphate, betamethasone sodium phosphate and acetate, betamethasone valerate, clobetasol proprionate, clocortolone pivalate, cortisol (hydrocortisone), cortisol (hydrocortisone) acetate, cortisol (hydrocortisone) butyrate, cortisol (hydrocortisone) cypionate, cortisol (hydrocortisone) sodium phosphate, cortisol (hydrocortisone) sodium succinate, cortisol (hydrocortisone) valerate, cortisone acetate, desonide, desoximetasone, dexamethasone, dexamethasone acetate, dexamethasone sodium phosphate, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, and triamcinolone hexacetonide, and combinations thereof. In some embodiments, romidepsin is administered in combination with dexamethasone.

In certain embodiments, romidepsin is administered in combination with an agent to treat gastrointestinal disturbances such as nausea, vomiting, and diarrhea. Such agents may include anti-emetics, anti-diarrheals, fluid replacement, electrolyte replacement, etc.

In some embodiments, romidepsin is administered in combination with electrolyte replacement or supplementation such as potassium, magnesium, and calcium, in particular, potassium and magnesium (see below).

In certain embodiments, romidepsin is administered in combination with an anti-arrhythmic agent.

In certain embodiments, romidepsin is administered in combination with a platelet booster, for example, an agent that increases the production of platelets.

In certain embodiments, romidepsin is administered in combination with an agent to boost the production of blood cells such as erythropoietin.

In some embodiments, romidepsin is administered in combination with an agent to prevent hyperglycemia.

In certain embodiments, romidepsin is not administered with another HDAC or DAC inhibitor.

### Electrolyte Supplementation

In accordance with the present invention, it may be desirable to administer electrolyte supplementation to subjects receiving romidepsin therapy. Individuals with low electrolyte levels (e.g., low potassium and/or magnesium levels) are susceptible to development of unwanted side effects if administered romidepsin therapy (see, for example, U.S. Ser. No. 11/759,471, published under U.S. Pub. No. US2008/0124403, incorporated herein by reference). Such patients may be particularly susceptible to development of cardiac repolarization effects, including QTc prolongation (though potentially with no significant cardiac function changes), and/or cardiac dysrhythmias. Particular abnormalities that may be observed include an increase in QTc interval and/or abnormalities of the ST segment (e.g., ST segment depression) and/or the T-wave (e.g., T-wave flattening) on ECG.

According to the present invention, an individual with a potassium serum concentration below about 3.5 mmol/L (3.5 mEq/L) and/or a serum magnesium concentration below about 0.8 mml/L (1.95 mEq/L) suffers an increased risk of developing cardiac repolarization effects and/or dysrhythmias.

Serum concentrations of potassium are generally considered to be “normal” when they are within the range of about 3.5-5.5 mEq/L or about 3.5-5.0 mEq/L. According to the present invention, it is often desirable to ensure that an individuals' serum potassium concentration is within these ranges prior to (and/or during) administration of romidepsin therapy.

Serum concentrations of magnesium are generally considered to be “normal” when they are within the range of about 1.5-2.5 mEq/L or about 1.5-2.2 mEq/L or about 1.25-2.5 mEq/L or about 1.25-2.2 mEq/L. According to the present invention, it is often desirable to ensure that an individual's serum magnesium concentration is within these ranges prior to (and/or during) administration of romidepsin therapy.

In some embodiments of the invention, an individual's serum potassium and/or magnesium concentration(s) is/are at the high end of the normal range prior to (and/or during) administration of romidepsin therapy. For example, in some embodiments, an individual's serum potassium concentration is at least about 3.8 mEq/L, 3.9 mEq/L, 4.0 mEq/L, or more prior to and/or during administration of romidepsin therapy. In some embodiments, care is taken not to increase serum potassium concentration above about 5.0 mEq/L, 5.2 mEq/L, or 5.5 mEq/L. In some embodiments, an individual's serum magnesium concentration is at least about 1.9 mEq/L or more prior to and/or during administration of romidepsin therapy. In some embodiments, care is taken not to increase magnesium concentration above about 2.5 mEq/L.

In some embodiments of the present invention, an individual's serum potassium concentration is at least about 3.5 mEq/L (in some embodiments at least about 3.8 mEq/L, 3.9 mEq/L, 4.0 mEq/L, or above) and the individual's serum magnesium concentration is at least about 1.85 mEq/L (in some embodiments at least about 1.25 mEq/L, 1.35 mEq/L, 1.45 mEq/L, 1.55 mEq/L, 1.65 mEq/L, 1.75 mEq/L, 1.85 mEq/L, 1.95 mEq/L, or above) prior to and/or during administration of romidepsin therapy.

In some embodiments of the invention, electrolyte levels (e.g., potassium and/or magnesium levels, optionally calcium levels) are assessed more than once during the course of romidepsin therapy; in some embodiments, different assessments are separated by a regular interval (e.g., 0.5 days or less, 1 day, 2 days, 3 days, 4 days, 5 days, 6 days, 7 days, 8 days, 9 days, 10 days, 11 days, 12 days, 13 days, 14 days, 1 month, 2 months, 3 months, 4 months, 5 months, 6 months, etc.). In some embodiments, electrolyte levels are assessed prior to each administration of romidepsin.

According to the present invention, an individual's serum potassium and/or magnesium and/or other electrolyte concentration(s) may be assessed by any available means. For example, samples may be collected from venous or arterial blood and processed for plasma or serum analysis. In some embodiments, venous sampling is utilized. Any available assay may be utilized for assessment. To give but a few specific examples, potassium may be measured by flame photometry, direct potentiometry (see, for example, Koch et al., Clin. Chem. 29:1090, 1983), enzymatic methods (e.g., by using tryptophanase, see, for example, Kimura et al., Clin. Chem. 38:44, 1992), colorimetric methods (e.g., using tetraphenyl borate), etc.; magnesium may be measured by complexometric titration, flame emission photometry, atomic absorption spectophotometry, other spectrophotometric techniques including enzymatic techniques and dye binding methods (e.g., Magnon dye binding and bichromatic absorbance, see, for example, Barbour et al., Clin. Chem. 34:2103, 1988; elimination of interference by bilirubin, see, for example, Rehak et al., Clin. Chem. 35:1031, 1989; etc.). In many embodiments, assays are performed in an automated clinical chemistry analyzer (e.g., the Abbott ARCHITECT®, etc.)

Where both potassium and magnesium levels are assessed, they may be assessed separately or together. Assessment of potassium and/or magnesium levels may be performed prior to, at the same time as, and/or after initiation of romidepsin therapy.

If an individual is determined to have serum potassium and/or magnesium concentration(s) that is/are below normal, or below the high end of normal as described herein, according to the present invention, potassium and/or magnesium supplementation is administered prior to, at the same time as, or after initiation of romidepsin therapy. In some embodiments, romidepsin therapy is suspended or delayed until serum potassium and/or magnesium levels are increased. In some embodiments, romidepsin therapy is suspended or delayed until serum potassium and/or magnesium levels are increased to within the normal range, or to within the upper end of the normal range. In some embodiments, romidepsin therapy is suspended or delayed until serum potassium concentration is above about 3.5 mEq/L; in some embodiments until serum potassium concentration is above about 3.8 mEq/L. In some embodiments, romidepsin therapy is suspended or delayed until serum magnesium concentration is above about 1.25 mEq/L; in some embodiments until serum magnesium concentration is above about 1.8 mEq/L; in some embodiments until serum magnesium concentration is above about 1.9 mEq/L. In some embodiments, romidepsin therapy is suspended or delayed until both serum potassium and serum magnesium concentrations are increased as described.

According to the present invention, electrolyte supplementation, which may be administered prior to, concurrently with, and/or subsequent to initiation of romidepsin therapy, may include potassium and/or magnesium supplementation. Alternatively or additionally, electrolyte supplementation may include supplementation of one or more electrolytes selected from the group consisting of sodium, potassium, chloride, calcium, magnesium, bicarbonate, phosphate, sulfate, and combinations thereof.

A variety of different potassium supplemental forms is available (see, for example, the web page at the following world wide web address: pdrhealth.com). For example, potassium supplements in the form of potassium chloride, potassium citrate, potassium gluconate, potassium bicarbonate, potassium aspartate and/or potassium orotate can readily be obtained.

High-potassium (up to 800 milligrams per serving), low-sodium vegetable juices are available. Some soft drinks are rich in potassium. Some soft drinks contain potassium gluconate which has a less bitter taste than some other potassium supplements. Salt substitutes are high in potassium.

Certain foods high in potassium such as raisins, figs, apricots, sardines, veal, bananas, avocado, and broccoli may be used as potassium supplements. Foods high in potassium may provide potassium that is easily bioavailable and/or may reduce gastrointestinal side effects associated with the administration of potassium salts. The potassium supplement may also be provided as part of a multivitamin.

Potassium is typically supplemented orally or intravenously, though other modes of delivery are within the scope of the present invention.

Certain commercially available forms of potassium supplements include, for example, potassium acetate (e.g., 2 mEq/mL or 4 mEq/mL for injection); potassium acetate (e.g., 75 mg, 95 mg, 99 mg, and 180 mg tablets and/or 2 mEq/mL, 10 mEq/50 mL, 20 mEq/50 mL, 10 mEq/100 mL, 20 mEq/100 mL, 30 mEq/100 mL, 40 mEq/100 mL for injection and/or 20 mEq/15 mL, 40 mEq/15 mL liquid and/or 20 mEq or 25 mEq powder for reconstitution, and/or 9 mEq, 10 mEq, or 20 mEq extended release tablets), and potassium gluconate (e.g., 486 mg, 500 mg, 550 mg, 595 mg, 610 mg, and 620 mg tablets).

A variety of different magnesium supplemental forms are also available. For example, supplements in the form of magnesium chloride, magnesium gluconate, magnesium lactate, magnesium oxide and/or magnesium sulfate can readily be obtained.

Certain foods high in magnesium such as artichoke, banana, figs, almonds, cashews, pine nuts, brazil nuts, beans, spinach, and tomatoes may be used as magnesium supplements. The magnesium supplement may also be provided as part of a multivitamin.

Certain commercially available forms of magnesium supplements include magnesium chloride (e.g., 200 mg/ml for injection, 535 mg extended release tablets), magnesium gluconate (3.25 mg/mL, 1000 mg/5 mL liquid; 500 mg tablet); magnesium lactate (84 mg extended release tablet); magnesium oxide (e.g., 140 mg, 600 mg capsules, powder, and/or 200 mg, 250 mg, 400 mg, 420 mg, and 500 mg tablets), magnesium sulfate (e.g., 40 mg/mL, 80 mg/mL, 125 mg/mL, 500 mg/mL for injection).

In some embodiments of the present invention, electrolyte supplementation is administered in an amount sufficient to reduce or delay onset of one or more cardiac toxicities of romidepsin therapy. In some embodiments, the electrolyte administration may also reduce one or more of nausea, vomiting, fatigue (lethargy, malaise, asthenia), increased creatine phospho kinase (CPK), hyperuricemia, hypocalcemia, hyperglycemia, fever, gastritis, diarrhea, abdominal pain, dehydration, weight loss, hypophosphatemia, hyponatremia, hypokalemia, hypomagnesemia, syncope, hypoxia, pleural effusion, hypotension, myocardial ischemia, increased cardiac troponin I, confusion, and/or myelosuppression, and combinations thereof.

In some embodiments, cardiac toxicities are selected from the group consisting of heart-rate corrected QT (QTc) interval prolongation, supraventricular arrhythmias (supraventricular tachycardia (SVT)/atrial fibrillation/flutter), and combinations thereof. Specifically, in some embodiments, QTc prolongation and/or other electrophysiological changes are reduced to normal values or ranges after electrolyte supplementation.

### Conditions to be Treated

The present invention provides methods and compositions relating to treatment of cell proliferative disorders, diseases or conditions. In general, cell proliferative disorders, diseases or conditions include a variety of conditions characterized by aberrant cell growth, preferably abnormally increased cellular proliferation. For example, cell proliferative disorders, diseases, or conditions include, but are not limited to, cancer, immune-mediated responses and diseases (e.g., transplant rejection, graft vs host disease, immune reaction to gene therapy, autoimmune diseases, pathogen-induced immune dysregulation, etc.), certain circulatory diseases, and certain neurodegenerative diseases.

In certain embodiments, the invention relates to methods of treating cancer. In general, cancer is a group of diseases which are characterized by uncontrolled growth and spread of abnormal cells. Examples of such diseases are carcinomas, sarcomas, leukemias, lymphomas and the like. In certain embodiments, the cancer is a hematological malignancy. In certain embodiments, the cancer is a solid tumor.

In certain embodiments the present invention relates to treatment of hematological malignancies. Manifestations of hematological malignancies can include circulating malignant cells as well as malignant masses. Hematological malignancies are types of cancers that affect the blood, bone marrow, and/or lymph nodes. Examples of hematological malignancies that may be treated using romidepsin include, but are not limited to: acute lymphoblastic leukemia (ALL), acute myelogenous leukemia (AML), chronic myelogenous leukemia (CML), chronic lymphocytic leukemia (CLL), hairy cell leukemia, Hodgkin's lymphoma, non-Hodgkin's lymphoma, cutaneous T-cell lymphoma (CTCL), peripheral T-cell lymphoma (PTCL), multiple myeloma, and myelodysplastic syndromes. In certain embodiments, romidepsin is used to treat multiple myeloma. In certain particular embodiments, the cancer is relapsed and/or refractory multiple myeloma. In other embodiments, romidepsin is used to treat chromic lymphocytic leukemia (CLL). In certain particular embodiments, the cancer is relapsed and/or refractory CLL. In other embodiments, romidepsin is used to treat chromic myelogenous leukemia (CML). In certain embodiments, romidepsin is used to treat acute lymphoblastic leukemia (ALL). In certain embodiments, romidepsin is used to treat acute myelogenous leukemia (AML). In certain embodiments, the cancer is cutaneous T-cell lymphoma (CTCL). In other embodiments, the cancer is peripheral T-cell lymphoma (PTCL). In certain embodiments, the cancer is a myelodysplastic syndrome.

In some embodiments of the present invention, cancers treated with romidepsin may include, without being limited to, leukemias and lymphomas such as cutaneous T-cell lymphomas (CTCL), peripheral T-cell lymphomas, lymphomas associated with human T-cell lymphotropic virus (HTLV) such as adult T-cell leukemia/lymphoma (ATLL), B-cell lymphoma, acute lymphocytic leukemia, acute nonlymphocytic leukemias, chronic lymphocytic leukemia, chronic myelogenous leukemia, acute myelogenous leukemia, Hodgkin's disease, non-Hodgkin's lymphomas, multiple myeloma, myelodysplastic syndromes, mesothelioma, common solid tumors of adults such as head and neck cancers (e.g., oral, laryngeal and esophageal), genitourinary cancers (e.g., prostate, bladder, renal, uterine, ovarian, testicular, rectal and colon), lung cancer, breast cancer, pancreatic cancer, melanoma and other skin cancers, stomach cancer, brain tumors, liver cancer and thyroid cancer, and/or childhood solid tumors such as brain tumors, neuroblastoma, retinoblastoma, Wilms' tumor, bone tumors, and soft-tissue sarcomas.

Alternatively or additionally, exemplary cancers that may be treated using romidepsin therapy, including combination therapy, include colon cancer, lung cancer, bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, etc.

In certain embodiments, romidepsin is used to treat pancreatic cancer. In certain embodiments, romidepsin is used to treat prostate cancer. In certain specific embodiments, the prostate cancer is hormone refractory prostate cancer.

In some particular embodiments, the invention relates to treatment of leukemias. For example, in some embodiments, the invention relates to treatment of chronic lymphocytic leukemia, chronic myelogenous leukemia, acute lymphocytic leukemia, acute myelogenous leukemia, and/or adult T cell leukemia/lymphoma. In certain embodiments, the invention relates to the treatment of AML. In certain embodiments, the invention relates to the treatment of ALL. In certain embodiments, the invention relates to the treatment of CML. In certain embodiments, the invention relates to the treatment of CLL.

In some embodiments, the invention relates to treatment of lymphomas. For example, in some embodiments, the invention relates to treatment of Hodgkin's or non-Hodgkin's (e.g., T-cell lymphomas such as peripheral T-cell lymphomas, cutaneous T-cell lymphomas, etc.) lymphoma.

In some embodiments, the invention relates to the treatment of multiple myeloma and/or myelodysplastic syndromes. In some embodiments, the invention relates to treatment of solid tumors. In some such embodiments the invention relates to treatment of solid tumors such as lung, breast, colon, liver, pancreas, renal, prostate, ovarian, and/or brain. In some embodiments, the invention relates to treatment of pancreatic cancer. In some embodiments, the invention relates to treatment of renal cancer. In some embodiments, the invention relates to treatment of prostate cancer. In some embodiments, the invention relates to treatment of sarcomas. In some embodiments, the invention relates to treatment of soft tissue sarcomas.

In some embodiments, the invention relates to methods of treating one or more immune-mediated responses and diseases. For example, in some embodiments, the invention relates to treatment of rejection following transplantation of synthetic or organic grafting materials, cells, organs, or tissue to replace all or part of the function of tissues, such as heart, kidney, liver, bone marrow, skin, cornea, vessels, lung, pancreas, intestine, limb, muscle, nerve tissue, duodenum, small-bowel, pancreatic-islet-cell, including xeno-transplants, etc.; treatment of graft-versus-host disease; autoimmune diseases, such as rheumatoid arthritis, systemic lupus erythematosus, thyroiditis, Hashimoto's thyroiditis, multiple sclerosis, myasthenia gravis, type I diabetes, juvenile-onset or recent-onset diabetes mellitus, uveitis, Graves' disease, psoriasis, atopic dermatitis, Crohn's disease, ulcerative colitis, vasculitis, auto-antibody mediated diseases, aplastic anemia, Evan's syndrome, autoimmune hemolytic anemia, and the like.

In some embodiments, the invention relates to methods of treating one or more infectious diseases causing aberrant immune response and/or activation, such as traumatic or pathogen induced immune dysregulation, including for example, that which are caused by hepatitis B and C infections, HIV, Staphylococcus aureus infection, viral encephalitis, sepsis, parasitic diseases wherein damage is induced by an inflammatory response (e.g., leprosy).

In some embodiments, the invention relates to treatment of graft vs host disease, rheumatoid arthritis, systemic lupus erythematosus, psoriasis, atopic dermatitis, Crohn's disease, ulcerative colitis, and/or multiple sclerosis.

Alternatively or additionally, in some embodiments, the invention relates to treatment of an immune response associated with a gene therapy treatment, such as the introduction of foreign genes into autologous cells and expression of the encoded product. In some embodiments, the invention relates to treatment of circulatory diseases, such as arteriosclerosis, atherosclerosis, vasculitis, polyarteritis nodosa and/or myocarditis.

In some embodiments, the invention relates to treatment of any of a variety of neurodegenerative diseases, a non-exhaustive list of which includes:
I. Disorders characterized by progressive dementia in the absence of other prominent neurologic signs, such as Alzheimer's disease; Senile dementia of the Alzheimer type; and Pick's disease (lobar atrophy);
II. Syndromes combining progressive dementia with other prominent neurologic abnormalities such as A) syndromes appearing mainly in adults (e.g., Huntington's disease, Multiple system atrophy combining dementia with ataxia and/or manifestations of Parkinson's disease, Progressive supranuclear palsy (Steel-Richardson-Olszewski), diffuse Lewy body disease, and corticodentatonigral degeneration); and B) syndromes appearing mainly in children or young adults (e.g., Hallervorden-Spatz disease and progressive familial myoclonic epilepsy);
III. Syndromes of gradually developing abnormalities of posture and movement such as paralysis agitans (Parkinson's disease), striatonigral degeneration, progressive supranuclear palsy, torsion dystonia (torsion spasm; dystonia musculorum deformans), spasmodic torticollis and other dyskinesis, familial tremor, and Gilles de la Tourette syndrome;
IV. Syndromes of progressive ataxia such as cerebellar degenerations (e.g., cerebellar cortical degeneration and olivopontocerebellar atrophy (OPCA)); and spinocerebellar degeneration (Friedreich's ataxia and related disorders);
V. Syndromes of central autonomic nervous system failure (Shy-Drager syndrome);
VI. Syndromes of muscular weakness and wasting without sensory changes (motomeuron disease such as amyotrophic lateral sclerosis, spinal muscular atrophy (e.g., infantile spinal muscular atrophy (Werdnig-Hoffman), juvenile spinal muscular atrophy (Wohlfart-Kugelberg-Welander) and other forms of familial spinal muscular atrophy), primary lateral sclerosis, and hereditary spastic paraplegia;
VII. Syndromes combining muscular weakness and wasting with sensory changes (progressive neural muscular atrophy; chronic familial polyneuropathies) such as peroneal muscular atrophy (Charcot-Marie-Tooth), hypertrophic interstitial polyneuropathy (Dejerine-Sottas), and miscellaneous forms of chronic progressive neuropathy;
VIII. Syndromes of progressive visual loss such as pigmentary degeneration of the retina (retinitis pigmentosa), and hereditary optic atrophy (Leber's disease).

In some embodiments, the neurodegenerative disease is Alzheimer's disease, Parkinson's disease, and/or Huntington's disease.

In some embodiments, the invention relates to treatment of disorders, diseases or conditions associated with chromatin remodeling.

Unless otherwise defined, all technical and scientific terms used herein are accorded the meaning commonly known to one of ordinary skill in the art. All publications, patents, published patent applications, and other references mentioned herein are hereby incorporated by reference in their entirety. The embodiments of the invention should not be deemed to be mutually exclusive and can be combined.

### EXEMPLIFICATION

The present invention will be better understood in connection with the following Example, which is intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1

### Accelerated Romidepsin Dosing

The present example summarizes clinical trial experience with accelerated dosing of romidepsin. At least fifteen patients were treated with accelerated dosing regimens. Two of these patients suffered from PTCL, one suffered from Graft Versus Host Disease (GVHD), six suffered from multiple myeloma, one suffered from Non-Hodgkin's Lymphoma, one suffered from melanoma, two suffered from breast cancer, one suffered from ovarian cancer, and one suffered from a Giant Cell tumor.

Tables 2 and 3 below summarize some of the relevant data. The electrocardiogram findings, lab abnormalities, adverse events, and serious adverse events in these 15 patients are consistent with the results seen in prior clinical trials utilizing romidepsin.

TABLE 2

| | | | Adverse Events | | | |
|---|---|---|---|---|---|---|
| | | Accelerated | reported directly | | | |
| Patient | Regimen | Dose(s) | after infusion | ECG | LAB | Notes |
| Male | 30.5 mg on | Day 1 of | None | Post infusion | No immediate | Patient was |
| 58 yrs old | days 1, 8, and | cycle 8 | | ECG | effect but | sent home the |
| 95.8 Kg | 15 of 28 day | | | asymptomatic - | hypocalcaemia | same day and |
| BSA 2.18 | cycle | | | showed | later | was followed |
| | | | | some lateral | | up with |
| | | | | ST depression | | phone call the |
| | | | | of 1 mm. | | next day. |
| | | | | ECG 2 hours | | |
| | | | | later showed | | |
| | | | | improvement | | |
| Male | 26 mg on | Day 15 of | Nausea, | Post infusion | Hypocalcaemia | |
| 65 years old | days 1, 8, and | cycle 8 | vomiting and | ECG - | noted | |
| 78 kg | 15 of 28 day | | diarrhoea, | peaked T | | |
| BSA: 1.85 m² | cycle | | heamaturia | waves | | |
| | | | | laterally and | | |
| | | | | subtle ST | | |
| | | | | depression | | |
| | | | | laterally but | | |
| | | | | otherwise | | |
| | | | | normal | | |
| | | | | ECG next day | | |
| | | | | was normal | | |
| Female | 13 mg on | Day 15 of | None | Post dose | No | Patient was |
| 66 years old | days 1, 8, and | cycle 2 | | ECGs taken | abnormalities | kept in the |
| | 15 of 28 day | | | on same day | | hospital |
| | cycle | | | and next day; | | overnight for |
| | | | | no | | observation |
| | | | | abnormalities | | and was |
| | | | | notes | | discharged |
| | | | | | | next day |

TABLE 3

| | | | # of | | | Clinically | |
|---|---|---|---|---|---|---|---|
| | Protocol | Accelerated | Accelerated | Related Adverse | ECG Findings | Significant Lab | Related |
| Patient | (Diagnosis) | Regimen | Doses | Events | (Machine Alerts) | Abnormalities | SAEs |
| Male, | GPI-06-0005 | 8 mg/m² on | 3 | Fatigue | Probable normal | None | None |
| 53 yrs old, | (NHL) | Days 1, 8, | | | variant; borderline | | |
| 87 Kg, | | and 15 of | | | lateral T wave | | |
| BSA 2.01 | | Cycle 2 and | | | changes; low QRS | | |
| | | beyond | | | voltages | | |
| Female, | GPI-06-0005 | 8 mg/m² on | 3 | Fatigue, | Nonspecific T wave | None | None |
| 52 yrs old, | (Melanoma) | Days 1, 8, | | vomiting | changes possibly due | | |
| 95 Kg, | | and 15 of | | | to myocardial | | |
| BSA 2.03 | | Cycle 2 and | | | ischemia; low QRS | | |
| | | beyond | | | voltages; sinus | | |
| | | | | | tachycardia; possible | | |
| | | | | | inferior infarction | | |
| Female, | GPI-06-0005 | 8 mg/m² on | 6 | Fatigue, | Nonspecific T wave | None | None |
| 46 yrs old, | (Breast | Days 1, 8, | | dysphagia, | changes; low QRS | | |
| 51 Kg, | Cancer) | and 15 of | | dizziness | voltages; | | |
| BSA 1.53 | | Cycle 2 and | | | tachycardia; possible | | |
| | | beyond | | | anterior infarction; | | |
| | | | | | left atrial abnormality | | |
| Female | GPI-06-0005 | 8 mg/m² on | 5 | Fatigue, | Left anterior | None | None |
| 77 yrs old, | (Breast | Days 1, 8, | | vomiting, | fascicular block; low | | |
| 87 Kg, | Cancer) | and 15 of | | anemia, | QRS voltages, | | |
| BSA 1.85 | | Cycle 2 and | | anorexia | nonspecific T wave | | |
| | | beyond | | | changes; possible left | | |
| | | | | | ventricular | | |
| | | | | | hypertrophy, possible | | |
| | | | | | anterior infarction; | | |
| | | | | | tachycardia; sinus | | |
| | | | | | arrhythmia | | |
| Female, | GPI-06-0005 | 12 mg/m² on | 14* | Not in | Possible interior and | Not in | Atrial |
| 57 yrs old, | (Ovarian | Days 1, 8, | | database^{#} | anterior infarction, | database^{#} | fibrillation |
| 85.9 Kg, | Cancer) | and 15 of | | | nonspecific T wave | | |
| BSA 1.93 | | Cycle 2 and | | | changes possibly due | | |
| | | beyond | | | to myocardial | | |
| | | | | | ischemia, low QRS | | |
| | | | | | voltages; possible | | |
| | | | | | WPW pattern, left | | |
| | | | | | ventricular | | |
| | | | | | hypertrophy; short | | |
| | | | | | PR interval | | |
| Male | GPI-06-0005 | 12 mg/m² on | 11* | Not in | Probable variant of | Not in | None |
| 64 yrs old, | (Giant | Days 1, 8, | | database^{#} | poor R wave | database^{#} | |
| 97.5 Kg, | Cell) | and 15 of | | | progression; | | |
| BSA 2.14 | | Cycle 2 and | | | nonspecific T waves | | |
| | | beyond | | | changes possibly due | | |
| | | | | | to myocardial | | |
| | | | | | ischemia; possible | | |
| | | | | | anterior infarction, | | |
| | | | | | low QRS voltages; | | |
| | | | | | sinus tachycardia | | |
| Female | GPI-08-0006 | 10 mg/m² on | 13 | Nausea, | Nonspecific T waves | Thrombocytopenia, | None |
| 72 yrs old, | (Multiple | Days 1, 8, | | vomiting, | changes possibly due | anemia, | |
| 76 Kg, | Myeloma) | and 15 of | | flushing, | to myocardial | neutropenia | |
| BSA 1.79 | | Cycle 1 and | | constipation, | ischemia; results | | |
| | | beyond | | headache, | consistent with | | |
| | | | | body aches, | baseline ECGs | | |
| | | | | abdominal | | | |
| | | | | pain, | | | |
| | | | | diarrhea, | | | |
| | | | | bloating, | | | |
| | | | | vein | | | |
| | | | | erythema | | | |
| Female | GPI-08-0006 | 10 mg/m² on | 2 | Flushing, | Sinus tachycardia, | Thrombocytopenia, | None |
| 71 yrs old, | (Multiple | Days 1, 8, | | cold feeling, | nonspecific ST | anemia, | |
| 70.3 Kg, | Myeloma) | and 15 of | | nausea, | changes; low QRS | neutropenia, | |
| BSA 1.68 | | Cycle 1 and | | vomiting, | voltages; junctional | increased | |
| | | beyond | | back pain, | depression prior to | serum | |
| | | | | canker sore, | baseline | creatinine, | |
| | | | | fatigue, UTI, | | hyperpcalcemia | |
| | | | | hypokalemia, | | | |
| | | | | hypophosphatemia | | | |
| Female | GPI-08-0006 | 10 mg/m² on | 1 | Nausea, | T wave inversion, | Thrombocytopenia, | Vomiting, |
| 68 yrs old, | (Multiple | Days 1, 8, | | vomiting, | low QRS voltages; | anemia, | headache, |
| 60.3 Kg, | Myeloma) | and 15 of | | headache, | reversed R wave | neutropenia, | dehydration |
| BSA 1.66 | | Cycle 1 and | | dehydration | progression | hypoalbuminemia, | |
| | | beyond | | | | hypokalemia | |
| Male | GPI-08-0006 | 10 mg/m² on | 4 | Nausea, | Nonspecific T wave | Thrombocytopenia, | None |
| 65 yrs old, | (Multiple | Days 1, 8, | | thrombocytopenia, | changes; ST | anemia, | |
| 103 Kg, | Myeloma) | and 15 of | | fatigue, vein | junctional depression | neutropenia, | |
| BSA 2.26 | | Cycle 1 and | | erythema, | | hypoalbuminemia | |
| | | beyond | | vomiting | | | |
| Male | GPI-08-0006 | 10 mg/m² on | 2 | Knee pain, | T wave changes, | Thrombocytopenia, | None |
| 67 yrs old, | (Multiple | Days 1, 8, | | headache, | conduction defect of | anemia, | |
| 89.8 Kg, | Myeloma) | and 15 of | | skin | right bundle branch | neutropenia, | |
| BSA 1.96 | | Cycle 1 and | | crawling, | block; low QRS | increased | |
| | | beyond | | cold feet, | spatial velocity; QTC | serum | |
| | | | | malaise, | close to 450 | creatinine | |
| | | | | bowel | | | |
| | | | | urgency, | | | |
| | | | | nausea, | | | |
| | | | | vomiting | | | |
| Male | GPI-08-0006 | 8 mg/m² on | 4 | Not in | None | None | None |
| 73 yrs old, | (Multiple | Days 1, 8, | | database^{#} | | | |
| 97.3 Kg, | Myeloma) | and 15 of | | | | | |
| BSA 2.1 | | Cycle 1 and | | | | | |
| | | beyond | | | | | |

*ongoing patient
^{#}Data had not yet been retrieved during a monitoring visit

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EQUIVALENTS

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

To give but a few examples, in the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is further understood that any listing of elements in Markush group format is not intended as a concession that individual elements are not patentably distinct from one another, but rather is intended only to simplify presentation of multiple alternatives.

It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included unless otherwise indicated. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For example, in certain embodiments of the invention the biologically active agent is not an anti-proliferative agent. For purposes of brevity, all of the embodiments in which one or more elements, features, purposes, or aspects is excluded are not set forth explicitly herein.

What is claimed is:

1. A method comprising the step of:
administering intravenously, over a time period less than about 1 hour, at least one unit dose of romidepsin to a subject having a proliferative disease.

2. The method of claim 1, wherein the time period is less than about 50 minutes.

3. The method of claim 1, wherein the time period is less than about 40 minutes.

4. The method of claim 1, wherein the time period is less than about 30 minutes.

5. The method of claim 1, wherein the time period is less than about 20 minutes.

6. The method of claim 1, wherein the step of administering is repeated such that at least two separate unit doses of romidepsin are administered intravenously and each dose is administered over a time period that is less than about an hour.

7. The method of claim 1, further comprising at least one step of administering a unit dose of romidepsin intravenously over a time period that exceeds about an hour.

8. The method of claim 1, wherein the step of administering is repeated on days 1, 8, and 15 of a 28-day cycle.

9. The method of claim 6, wherein the 28-day cycle is repeated for 2-10 times.

10. The method of claim 6, wherein the 28-day cycle is repeated for 2-7 times.

11. The method of claim 6, wherein the 28-day cycle is repeated for 2-5 times.

12. The method of claim 1, wherein the unit dose of romidepsin ranges from approximately 0.5 mg/m² to approximately 35 mg/m².

13. The method of claim 12, wherein the unit dose of romidepsin ranges from approximately 1 mg/m² to approximately 35 mg/m².

14. The method of claim 12, wherein the unit dose of romidepsin ranges from approximately 5 mg/m² to approximately 28 mg/m².

15. The method of claim 12, wherein the unit dose of romidepsin ranges from approximately 8 mg/m² to approximately 14 mg/m².

16. The method of claim 12, wherein the effective dose of romidepsin is approximately 30 mg/m².

17. The method of claim 12, wherein the unit dose of romidepsin is approximately 26 mg/m².

18. The method of claim 12, wherein the unit dose of romidepsin is approximately 13 mg/m².

19. The method of claim 12, wherein the unit dose of romidepsin is approximately 12 mg/m².

20. The method of claim 12, wherein the unit dose of romidepsin is approximately 10 mg/m².

21. The method of claim 12, wherein the unit dose of romidepsin is approximately 8 mg/m².

22. The method of claim 1, wherein the subject does not suffer a serious adverse event associated with the administration.

23. The method of claim 1, wherein:
the step of administering is repeated for a plurality of patients; and wherein such administration results in a rate of observed toxicities not materially worse than a standard rate of toxicities observed for administration of a comparable dosing regimen that differs only in that unit doses of romidepsin are administered intravenously over a time period of about 4 hours.

24. The method of claim 23, wherein the toxicities are selected from the group consisting of fatigue, hematological toxicities, cardiac toxicities, gastrointestinal toxicities, constitutional toxicities, and combinations thereof.

25. The method of claim 1, wherein the subject's QTc remains below about 500 msec within 48 hours after administration of the accelerated dose.

26. The method of claim 1, wherein the subject does not suffer a ventricular arrhythmia during the administering step.

27. The method of claim 1, wherein the subject does not suffer sinus tachycardia during the administering step.

28. The method of claim 1, wherein the subject does not suffer an atrial dysrhythmia during the administering step.

29. The method of claim 1, wherein the subject does not suffer ST or T-wave changes indicative of repolarization during the administering step.

30. The method of claim 1, wherein the romidepsin is of the formula:

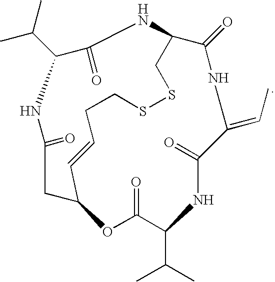

31. The method of claim 1, wherein the method comprises administering an anti-neoplastic agent together with the romidepsin.

32. The method of claim 1, wherein the method comprises administering a cytotoxic agent together with the romidepsin.

33. The method of claim 1, wherein the method comprises administering Gemcitabine together with the romidepsin.

34. The method of claim 1, wherein the method comprises administering at least one taxane together with the romidepsin.

35. The method of claim 1, wherein the method comprises administering a proteasome inhibitor together with the romidepsin.

36. The method of claim 35, wherein the proteasome inhibitor is selected from the group consisting of bortezomib (VELCADE®), peptide boronates, salinosporamide A (NPI-0052), lactacystin, epoxomicin (Ac(Me)-Ile-Ile-Thr-Leu-EX), MG-132 (Z-Leu-Leu-Leu-al), PR-171, PS-519, eponemycin, aclacinomycin A, CEP-1612, CVT-63417, PS-341 (pyrazylcarbonyl-Phe-Leu-boronate), PSI (Z-Ile-Glu(OtBu)-Ala-Leu-al), MG-262 (Z-Leu-Leu-Leu-bor), PS-273 (MNLB), omuralide (clasto-lactacystin-β-lactone), NLVS (Nip-Leu-Leu-Leu-vinyl sulfone), YLVS (Tyr-Leu-Leu-Leu-vs), dihydroeponemycin, DFLB (dansyl-Phe-Leu-boronate), ALLN (Ac-Leu-Leu-Nle-al), 3,4-dichloroisocoumarin, 4-(2-aminoethyl)-benzenesulfonyl fluoride, TMC-95A, gliotoxin, EGCG ((−)-epigallocatechin-3-gallate), YU101 (Ac-hFLFL-ex), and combinations thereof.

37. The method of claim 1, wherein the method further comprises administering a steroidal agent to the subject.

38. The method of claim 37, wherein the steroidal agent is selected from the group consisting of alclometasone diproprionate, amcinonide, beclomethasone diproprionate, betamethasone, betamethasone benzoate, betamethasone diproprionate, betamethasone sodium phosphate, betamethasone sodium phosphate and acetate, betamethasone valerate, clobetasol proprionate, clocortolone pivalate, cortisol (hydrocortisone), cortisol (hydrocortisone) acetate, cortisol (hydrocortisone) butyrate, cortisol (hydrocortisone) cypionate, cortisol (hydrocortisone) sodium phosphate, cortisol (hydrocortisone) sodium succinate, cortisol (hydrocortisone) valerate, cortisone acetate, desonide, desoximetasone, dexamethasone, dexamethasone acetate, dexamethasone sodium phosphate, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, and triamcinolone hexacetonide, and combinations thereof.

39. The method of claim 1, wherein the proliferative disease is a benign neoplasm.

40. The method of claim 1, wherein the proliferative disease is cancer.

41. The method of claim 40, wherein the cancer is a solid tumor.

42. The method of claim 40, wherein the cancer is prostate cancer.

43. The method of claim 40, wherein the cancer is a hematological cancer.

44. The method of claim 40, wherein the cancer is a leukemia.

45. The method of claim 40, wherein the cancer is a lymphoma.

46. The method of claim 40, wherein the cancer is cutaneous T-cell lymphoma (CTCL).

47. The method of claim 40, wherein the cancer is peripheral T-cell lymphoma (PTCL).

48. The method of claim 40, wherein the cancer is multiple myeloma.

49. The method of claim 40, wherein the cancer is selected from the group consisting of non-Hodgkin's lymphoma, Hodgkin's lymphoma, a lymphoproliferative malignancy, plasma cell-derived cancer, chronic lymphocytic leukemia (CLL), acute myelogenous leukemia (AML), and acute lymphoid leukemia (ALL).

50. The method of claim 40, wherein the cancer is a relapsed or refractory cancer.

51. The method of claim 1, further comprising administering electrolyte supplementation to the subject.

52. A method of treating a patient in need of a histone deacetylase inhibitor treatment, the method comprising:
administering intravenously, over a time period less than about one hour, a unit dose of romidepsin to the patient.

53. The method of claim 52, wherein the time period is less than 50 minutes.

54. The method of claim 52, wherein the time period is less than 40 minutes.

55. The method of claim 52, wherein the time period is no less than 30 minutes.

56. The method of claim 52, wherein the time period is no less than 20 minutes.
